(19) United States
(12) Patent Application Publication
(10) Pub. No.: US 2012/0302749 A1
(43) Pub. Date: Nov. 29, 2012

(54) PROCESSES FOR THE PREPARATION OF ERLOTINIB HYDROCHLORIDE FORM A AND ERLOTINIB HYDROCHLORIDE FORM B

(75) Inventors: Balaguru Murugesan, Coimbatore (IN); Anandam Vempali, Nellore (IN); Swargam Sathyanarayana, Karim Nagar (IN); Rajesh Kumar Thaper, Jammu (IN); Mohan Prasad, Gurgaon (IN)

(73) Assignee: RANBAXY LABORATORIES LIMITED, New Delhi, Delhi (IN)

(21) Appl. No.: 13/509,031

(22) PCT Filed: Nov. 12, 2010
(86) PCT No.: PCT/IB10/55155

§ 371 Date: Jun. 22, 2012

(30) Foreign Application Priority Data
Nov. 12, 2009 (IN) 2338/DEL/2009
Dec. 31, 2009 (IN) 2754/DEL/2009

(51) Int. Cl. C07D 239/94

(52) U.S. Cl. 544/293

(57) ABSTRACT

The present invention relates to processes for the preparation of erlotinib hydrochloride Form A and erlotinib hydrochloride Form B. (I).

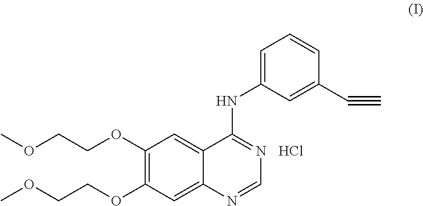

### FIELD OF THE INVENTION

The present invention relates to processes for the preparation of erlotinib hydrochloride Form A and erlotinib hydrochloride Form B.

### BACKGROUND OF THE INVENTION

Erlotinib hydrochloride of Formula I chemically, N-(3-ethynylphenyl)-6,7-bis(2-methoxyethoxy)quinazolin-4-amine hydrochloride, marketed under the brand name Tarceva® in United States is indicated for the treatment of patients with locally advanced or metastatic non-small cell lung cancer after failure of at least one prior chemotherapy regimen and in combination with gemcitabine is indicated for the first-line treatment of patients with locally advanced, unresectable or metastatic pancreatic cancer.

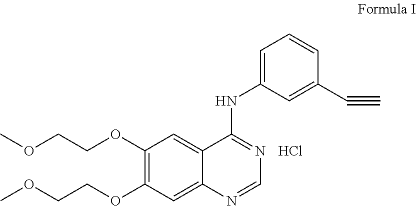

U.S. Pat. No. 5,747,498 (hereinafter “'498 patent”) provides a process for preparation of erlotinib hydrochloride. According to Example 20 of the US '498 patent, erlotinib free base was dissolved in a minimum volume of chloroform, diluted with several volumes of ether, and then titrated with 1M hydrochloric acid in ether to precipitate erlotinib hydrochloride. However, the '498 patent makes no reference to the existence of specific polymorphic form of erlotinib hydrochloride.

U.S. Pat. No. 6,900,221 (hereinafter “'221 patent”) mentions two crystalline forms of erlotinib hydrochloride designated as polymorph Form A and polymorph Form B. The '221 patent further mentions, in column 8, paragraph 45, that erlotinib hydrochloride disclosed in the '498 patent actually comprised a mixture of polymorphs A and B. According to the '221 patent erlotinib hydrochloride can be obtained in polymorphic Form A or in a mixture of polymorph A and B, by treating the filtrate containing 3-ethylaniline in toluene with 4-chloro-6,7-bis-(2-methoxyethoxy)-quinazoline and acetonitrile to reflux temperature, cooling the reaction mass to between 19° C. to 25° C. and isolating erlotinib hydrochloride in polymorph Form A or in mixture of polymorph A and B. Further, the '221 patent provides the production of polymorph A is favored by the reduction of the amount of acetonitrile relative to toluene, and particularly favored, if isopropanol is used in place of acetonitrile.

According to the '221 patent erlotinib hydrochloride can be obtained in polymorphic Form B by refluxing (˜80° C.) erlotinib hydrochloride (polymorph A or mixture of Form A and Form B), 2B-ethanol and water so as to form a solution, cooling the solution to between 65° C. and 70° C., clarifying the solution by filtration, cooling the solution to between 50° C. and 60° C. with low speed agitation followed by granulation of the precipitate, further cooling the mixture to between 0° C. and 5° C. followed by granulation of the precipitate and isolating erlotinib hydrochloride in polymorph Form B by filtration.

U.S. Pat. No. 7,148,231 provides a process for the preparation of erlotinib hydrochloride Form E by refluxing 4-chloro-6,7-bis(2-methoxyethoxy)quinazoline suspended in (α,α,α)-trifluorotoluene, 3-ethynylaniline dissolved in (α,α,α)-trifluorotoluene and hydrochloric acid (37%), after completion of the reaction, cooling the resulting suspension to room temperature, filtering and washing the isolated crystals of erlotinib hydrochloride with ethanol and drying at 60° C./10 mbar overnight to give erlotinib hydrochloride Form E.

U.S. Publication No. 2006/0154941 A1 provides a process for preparing amorphous form of erlotinib hydrochloride by dissolving crystalline erlotinib hydrochloride in methanol, ethanol or a mixture of ethanol and water to complete dissolution followed by removal of the solvent from the reaction mixture using distillation or spray drying to give amorphous erlotinib hydrochloride.

U.S. Publication No. 2008/0058355 A1 provides a process for preparation of erlotinib hydrochloride by adding erlotinib monohydrate Form I dissolved in acetone and 2-propanol with 5-6 N hydrochloric acid (>1 equivalent of hydrochloric acid) with continuous stirring, isolating the solid by filtration, and air drying overnight at room temperature to give erlotinib hydrochloride in a mixture of Form A and Form B.

U.S. Pat. No. 6,476,040 B1 (hereinafter “'040 patent”) provides a process for the preparation of erlotinib hydrochloride by treating erlotinib free base in 2-propanol, butan-1-ol, butan-2-ol or 2-methoxyethanol with concentrated hydrochloric acid to give crystalline erlotinib hydrochloride. However, the '040 patent makes no reference to the existence of specific polymorphic form of erlotinib hydrochloride except a melting point of 226° C. to 229° C. in Example 4.

U.S. Publication No. 2008/0167327 A 1 (hereinafter “'327 application”) provides a process for the preparation of erlotinib hydrochloride hemihydrate Form I by dissolving anhydrous erlotinib hydrochloride in demi-water at reflux, leaving the hot solution unagitated at 4° C. for 28 days during which slow crystallisation occurred, isolating the solid and air drying overnight at ambient temperature to give off-white to pale beige bunches of fibre-like crystals of erlotinib hydrochloride hemihydrate Form I.

The '327 application further provides a process for the preparation of erlotinib hydrochloride hemihydrate Form II by dissolving anhydrous erlotinib hydrochloride in demi-water at reflux, followed by adding seeds of erlotinib hydrochloride hemihydrate Form I, leaving the solution unagitated at 4° C. for 28 days unagitated at 4° C. for 4 days, during which slow crystallization occurs, isolating the solid and air drying overnight at ambient temperature to give pale yellow lumps of sticky powder of erlotinib hydrochloride hemihydrate Form II.

PCT Publication No. WO 2007/060691 A2 (hereinafter “'691 application”) provides a process for the preparation of erlotinib hydrochloride by treating erlotinib base dissolved in acetone or acetonitrile with isopropanolic hydrochloride at reflux temperatures, maintained for one hour, followed by cooling to 25° C. to 30° C. and isolating erlotinib monohydrochloride as a white solid.

WO 2008/102369 Al (hereinafter “'369 application”) provides a process for the preparation of Form M of erlotinib hydrochloride by treating erlotinib base in methanol with a solution of hydrogen chloride in dry methanol or isopropanol. The '369 application also provides a process for the preparation of Form N of erlotinib hydrochloride by treating erlotinib base in isopropanol with isopropanolic hydrogen chloride. The '369 application further provides a process for the preparation of Form P of erlotinib hydrochloride by treating erlotinib base in methylene chloride with isopropanolic hydrogen chloride.

WO 2009/024989 A2 provides process for preparing crystalline erlotinib hydrochloride polymorph Form A substantially free of polymorph B which involves dissolving erlotinib free base in methyl isobutyl ketone or isopropyl acetate treating the solution with 7% ethyl acetate hydrogen chloride.

WO 2009/025876 A2 (hereinafter “'876 application”) provides a process for the preparation of erlotinib hydrochloride Form F and erlotinib hydrochloride Form G by treating erlotinib base dissolved in a solvent selected from 1,3-dioxalane, butanol with hydrogen chloride selected from concentrated hydrochloric acid, aqueous hydrogen chloride in butanol or hydrogen chloride in ether.

WO 2009/025873 A2 (hereinafter “'873 application”) provides a process for the preparation of erlotinib hydrochloride Form A which involves crystallizing erlotinib hydrochloride from a solvent selected from the group consisting of: toluene, a mixture of toluene and methanol, methylal, tert butyl methylether (“TBME”), ethylacetate at 0° C., n-butanol, mixture of n-butanol and water, methylisobutyl ketone (“MIBK”), s-butanol, a mixture of s-butanol and water, n-propanol, 2-propanol, methoxyethanol, a mixture of methoxyethanol and water, ethanol, a mixture of 1,3-dioxolane and methanol, a mixture of 1,3-dioxolane and water and a mixture of butanone and water; wherein the mixture of 1,3-dioxolane and water has about 2% to about 3% v/v of water, the mixture of 1,3-dioxolane and methanol has about 10% v/v of methanol, the mixture of n-butanol and water has about 1% to about 2% v/v of water, the mixture of s-butanol and water has about 1% to about 2% v/v of water, the mixture of methoxyethanol and water has about 1% to about 2% v/v of water, and the mixture of toluene and methanol has about 2% v/v of methanol.

The '873 application also provides a process for the preparation of erlotinib hydrochloride Form B which involves crystallizing erlotinib hydrochloride from a solvent selected from the group consisting of: dichloromethane (“DCM”), diethylether, isopropyl acetate, methanol, mixture of n-butanol and water, mixture of s-butanol and water, mixture of methoxyethanol and water, mixture of 1,3-dioxolane and methanol, and mixture of 1,3-dioxolane and water, wherein the mixture of 1,3-dioxolane and water has about 5% to about 10% v/v of water, the mixture of 1,3-dioxolane and methanol has about 20% to about 40% v/v of methanol, mixture of n-butanol and water has about 5% to about 10% v/v of water, the mixture of s-butanol and water has about 10% v/v of water and the mixture of methoxyethanol and water has about 10% v/v of water.

The '873 application further provides a process for the preparation of erlotinib hydrochloride Form B which involves slurring crystalline erlotinib hydrochloride Form A in a solvent selected from the group consisting of: methanol, mixture of 1,3-dioxolane and water, n-heptane, and diethyl ether and mixtures thereof, wherein the mixture of 1,3-dioxolane and water has about 5% to about 10% v/v of water.

IP.Com document ID 000180601D provides a process for preparation of erlotinib hydrochloride having a characteristic peaks at 5.7, 9.8, 10.4, 11.4, 22.8, 28.0, 28.6, 29.3, 29.6 and 34.5±0.2 degrees 2-theta by treating erlotinib base dissolved in 1,3-dioxolane/H₂O (98:2) with 37% hydrogen chloride at 62° C. to 65° C. to give erlotinib hydrochloride.

PCT Publication Nos. WO 031066602A1, WO 2009/007984 A2, WO 2008/122776 A2, WO 2007/138612 A2, WO 2007/138613 A2, WO 2008/000418 A2 provides various processes for the preparation of erlotinib and its salts.

Solvent medium and mode of isolation play very important roles in obtaining a polymorphic form over another.

Since erlotinib hydrochloride constitutes an important therapeutic agent, additional and improved ways of preparing erlotinib hydrochloride are of value to the pharmaceutical science. Thus, there is a need in the development of consistent, commercially viable processes for preparing erlotinib hydrochloride Form A and erlotinib hydrochloride Form B, which are safer, less time consuming and/or provide products of better purity.

### SUMMARY OF THE INVENTION

One aspect of the present invention provides a process for preparing erlotinib hydrochloride Form A which comprises of:
a) providing a mixture comprising erlotinib and a solvent selected from acetone, dichloromethane or a mixture thereof;
b) treating the mixture obtained in step a) with hydrogen chloride gas; and
c) isolating crystalline erlotinib hydrochloride Form A.

Another aspect of the present invention provides a process for preparing erlotinib hydrochloride Form A which comprises of:
a) providing a mixture comprising erlotinib and a solvent selected from dichloromethane, ether or mixture thereof;
b) treating the mixture obtained in step a) with 4% hydrogen chloride in ether; and
c) isolating crystalline erlotinib hydrochloride Form A.

Yet another aspect of the present invention provides a process for preparing erlotinib hydrochloride Form A which comprises of:
a) providing a mixture comprising erlotinib and ethyl acetate;
b) treating the mixture obtained in step a) with hydrogen chloride gas at about 40° C. to about 70° C.; and
c) isolating crystalline erlotinib hydrochloride Form A.

Another aspect of the present invention provides a process for preparing erlotinib hydrochloride Form B which comprises of:
a) providing a mixture comprising erlotinib and acetonitrile;
b) treating the mixture obtained in step a) with hydrogen chloride gas; and
c) isolating crystalline erlotinib hydrochloride Form B.

Still another aspect of the present invention provides a process for preparing erlotinib hydrochloride Form B which comprises of:
a) dissolving erlotinib hydrochloride in a solvent mixture selected from acetone/water and acetonitrile/water;
b) cooling the solution obtained in step a); and
c) isolating crystalline erlotinib hydrochloride Form B.

### DETAILED DESCRIPTION OF THE INVENTION

One aspect of the present invention provides a process for preparing erlotinib hydrochloride Form A which comprises of:
a) providing a mixture comprising erlotinib and a solvent selected from acetone, dichloromethane or a mixture thereof;
b) treating the mixture obtained in step a) with hydrogen chloride gas; and
c) isolating crystalline erlotinib hydrochloride Form A.

Erlotinib prepared by any method known in the art can be used as starting material. Step a) of providing a mixture of erlotinib includes dissolving erlotinib in a solvent selected from acetone, dichloromethane or a mixture thereof, or obtaining an existing solution from a previous processing step of erlotinib in acetone, dichloromethane, or a mixture thereof.

In one embodiment, the mixture is optionally stirred at a temperature of below about reflux temperature of the solvent used for at least 2 minutes to about 2 hours, preferably at about 20° C. to about 35° C. for about 2 minutes to about 1 hour, and more preferably at about 25° C. to about 30° C. for about 5 minutes to about 15 minutes.

In another embodiment, the mixture is further cooled at a temperature of about 0° C. to about 24° C., preferably at about 15° C. to about 20° C.

The mixture obtained in step a) is treated with hydrogen chloride gas. The hydrogen chloride gas used is optionally anhydrous hydrogen chloride gas and may be prepared freshly by adding sulphuric acid into a mixture of sodium chloride in concentrated hydrochloric acid.

In one embodiment, the mixture obtained in step a) is treated with hydrogen chloride gas at a temperature of about 0° C. to about 24° C., preferably at about 15° C. to about 20° C., optionally, until crystallization appears.

In another embodiment, the temperature of the reaction mass is raised to a temperature of about 20° C. to about 35° C., preferably, at about 25° C. to about 30° C. and stirred for about 1 hour to about 24 hours, preferably, for about 3 hours to about 4 hours.

The isolation of crystalline erlotinib hydrochloride Form A in step c) may be carried out by filtration, solvent removal, layer separation, centrifugation, concentration, distillation, or a combination thereof.

The crystalline erlotinib hydrochloride Form A obtained may be washed with an organic solvent selected from acetone, dichloromethane, or a mixture thereof.

The crystalline erlotinib hydrochloride Form A obtained may be further dried in, for example, in Vacuum Tray Dryer.

Drying can be carried out under reduced pressure until the residual solvent content reduces to the desired amount such as an amount that is within the limits given by the International Conference on Harmonization on Technical Requirements for Registration of Pharmaceuticals for Human Use (“ICH”) guidelines.

In one embodiment, the drying is carried out at atmospheric pressure or reduced pressures, at temperatures such as about 25° C. to about 70° C. The drying can be carried out for any desired time period that achieves the desired result, such as for a time period of about 1 hour to about 20 hours.

Another aspect of the present invention provides a process for preparing erlotinib hydrochloride Form A which comprises of:
a) providing a mixture comprising erlotinib and a solvent selected from dichloromethane, ether or mixture thereof;
b) treating the mixture obtained in step a) with 4% hydrogen chloride in ether; and
c) isolating crystalline erlotinib hydrochloride Form A.

Erlotinib prepared by any method known in the art can be used as starting material. Step a) of providing a mixture of erlotinib includes dissolving erlotinib in a solvent selected from dichloromethane, ether or a mixture thereof, or obtaining an existing solution from a previous processing step of erlotinib in dichloromethane, ether or a mixture thereof.

In one embodiment, the mixture is optionally stirred at a temperature of below about reflux temperature of the solvent used for at least 2 minutes to about 2 hours, preferably, at about 20° C. to about 35° C. from about 2 minutes to about 1 hour, and still more preferably, at about 25° C. to about 30° C. from about 5 minutes to about 15 minutes.

In another embodiment, the mixture is further cooled at a temperature of below about 0° C. to about 24° C., preferably at about 15° C. to about 20° C.

The mixture obtained in step a) is treated with 4% hydrogen chloride in ether.

In one embodiment the mixture obtained in step a) is treated with 4% hydrogen chloride in ether at a temperature of about 0° C. to about 24° C., preferably at about 15° C. to about 20° C.

In another embodiment, the temperature of the reaction mass is raised to a temperature of about 20° C. to about 35° C., preferably, at about 25° C. to about 30° C. and stirred for about 1 hour to about 24 hours, preferably, for about 3 hours to about 4 hours.

The isolation of crystalline erlotinib hydrochloride Form A in step c) may be carried out by filtration, solvent removal, layer separation, centrifugation, concentration, distillation, or a combination thereof.

The crystalline erlotinib hydrochloride Form A obtained may be washed with an organic solvent selected from dichloromethane, ether or a mixture thereof.

The crystalline erlotinib hydrochloride Form A obtained may be further dried in, for example, in Vacuum Tray Dryer.

Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying is carried out at atmospheric pressure or reduced pressures, at temperatures such as about 25° C. to about 70° C. The drying can be carried out for any desired time period that achieves the desired result, such as for a time period of about 1 hour to about 20 hours.

Yet another aspect of the present invention provides a process for preparing erlotinib hydrochloride Form A which comprises of:
a) providing a mixture comprising erlotinib and ethyl acetate;
b) treating the mixture obtained in step a) with hydrogen chloride gas at about 40° C. to about 70° C.; and
c) isolating crystalline erlotinib hydrochloride Form A.

Erlotinib prepared by any method known in the art can be used as starting material. Step a) of providing a mixture of erlotinib includes dissolving erlotinib in ethyl acetate or obtaining an existing solution from a previous processing step of erlotinib in ethyl acetate.

The mixture obtained in step a) is treated with hydrogen chloride gas. The hydrogen chloride gas used is optionally anhydrous hydrogen chloride gas and may be prepared freshly by adding sulphuric acid into a mixture of sodium chloride in concentrated hydrochloric acid.

In one embodiment, the mixture obtained in step a) is treated with hydrogen chloride gas at a temperature of about 40° C. to about 70° C., preferably, at about 55° C. to about 60° C., optionally till crystallization appears.

In another embodiment, the temperature of the reaction mass is cooled to a temperature of about 20° C. to about 35° C., preferably, at about 25° C. to about 30° C. and stirred for about 1 hour to about 24 hours, preferably, for about 3 hours to about 4 hours.

The isolation of crystalline erlotinib hydrochloride Form A in step c) may be carried out by filtration, solvent removal, layer separation, centrifugation, concentration, distillation, or a combination thereof.

The crystalline erlotinib hydrochloride Form A obtained may be washed with an organic solvent, such as, ethyl acetate.

The crystalline erlotinib hydrochloride Form A obtained may be further dried in, for example, in Vacuum Tray Dryer.

Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

Another aspect of the present invention provides a process for preparing erlotinib hydrochloride Form B which comprises of:
a) providing a mixture comprising erlotinib and acetonitrile;
b) treating the mixture obtained in step a) with hydrogen chloride gas; and
c) isolating crystalline erlotinib hydrochloride Form B.

Erlotinib prepared by any method known in the art can be used as starting material. Step a) of providing a mixture of erlotinib includes dissolving or slurring erlotinib in acetonitrile or obtaining an existing solution from a previous processing step of erlotinib in acetonitrile.

In one embodiment, the mixture is optionally stirred at a temperature of below about reflux temperature of the solvent used for at least 2 minutes to about 2 hours, preferably, at about 20° C. to about 35° C. for about 2 minutes to about 1 hour, and still more preferably, at about 25° C. to about 30° C. for about 5 minutes to about 15 minutes.

The mixture obtained in step a) is treated with hydrogen chloride gas. The hydrogen chloride gas used is optionally anhydrous hydrogen chloride gas and may be prepared freshly by adding sulphuric acid into a mixture of sodium chloride in concentrated hydrochloric acid.

In one embodiment, the mixture obtained in step a) is treated with hydrogen chloride gas at a temperature of about 0° C. to about 80° C., preferably at about 25° C. to about 30° C., optionally until crystallization appears.

The mixture obtained in step b) is stirred at a temperature from about 20° C. to 40° C. preferably at about 25° C. to about 30° C. for a time period of 1 hour to 24 hours, preferably for about 5 hours.

The isolation of crystalline erlotinib hydrochloride Form B in step c) may be carried out by filtration, solvent removal, layer separation, centrifugation, concentration, distillation, or a combination thereof.

The crystalline erlotinib hydrochloride Form B obtained may be washed with an organic solvent such as acetonitrile.

The crystalline erlotinib hydrochloride Form B obtained may be further dried in, for example, in Vacuum Tray Dryer.

Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying is carried out at atmospheric pressure or reduced pressures, at temperatures such as about 25° C. to about 70° C. The drying can be carried out for any desired time period that achieves the desired result, such as, for a time period of about 1 hour to about 24 hours.

Still another aspect of the present invention provides a process for preparing erlotinib hydrochloride Form B which comprises of:
a) dissolving erlotinib hydrochloride in a solvent mixture selected from acetone/water and acetonitrile/water;
b) cooling the solution obtained in step a); and
c) isolating crystalline erlotinib hydrochloride Form B.

Erlotinib hydrochloride prepared by any method known in the art can be used as starting material. Step a) of dissolving erlotinib hydrochloride includes dissolving erlotinib hydrochloride in a solvent mixture selected from acetone/water or acetonitrile/water at a temperature of about 45° C. to about 80° C. for about 2 minutes to about 1 hour, and still more preferably, at about 55° C. to about 75° C. for about 5 minutes to about 15 minutes.

In one embodiment, cooling the solution obtained in step a) includes cooling the obtained solution in step a) to a temperature of about 15° C. to about 40° C., preferably, at 25° C. to about 30° C. and stirring the reaction mixture for a time period of about 1 hour to about 24 hours, preferably, for about 15 hours to about 18 hours, more preferably, for about 17 hours. The reaction mass obtained is optionally further cooled to a temperature of about 0° C. to about 14° C., preferably, to a temperature of about 0° C. to about 5° C. and stirred at that temperature, preferably, at about 0° C. to about 5° C. for a period of about 1 hour to about 4 hours, preferably, for a period of about 2 hours to about 3 hours.

The isolation of crystalline erlotinib hydrochloride Form B in step c) may be carried out by filtration, solvent removal, layer separation, centrifugation, concentration, distillation, or a combination thereof.

The crystalline erlotinib hydrochloride Form B obtained may be washed with a solvent mixture selected from acetone/water or acetonitrile/water.

The crystalline erlotinib hydrochloride Form B obtained may be further dried in, for example, in Vacuum Tray Dryer.

Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying is carried out at atmospheric pressure or reduced pressures, at temperatures, such as about 25° C. to about 70° C. The drying can be carried out for any desired time period that achieves the desired result, such as for a time period of about 1 hour to about 24 hours.

### BRIEF DESCRIPTION OF THE DRAWINGS

FIG. 1a and FIG. 1b depicts XRPD of crystalline erlotinib hydrochloride Form A and the associated values, respectively, prepared as per Example 1.

FIG. 2a and FIG. 2b depicts XRPD of crystalline erlotinib hydrochloride Form A and the associated values, respectively, prepared as per Example 2.

FIG. 3a and FIG. 3b depicts XRPD of crystalline erlotinib hydrochloride Form A and the associated values, respectively, prepared as per Example 3.

FIG. 4a and FIG. 4b depicts XRPD of crystalline erlotinib hydrochloride Form A and the associated values, respectively, prepared as per Example 4.

FIG. 5a and FIG. 5b depicts XRPD of crystalline erlotinib hydrochloride Form

A and the associated values, respectively, prepared as per Example 5.

FIG. 6a and FIG. 6b depicts XRPD of crystalline erlotinib hydrochloride Form B and the associated values, respectively, prepared as per Example 6.

FIG. 7a and FIG. 7b depicts XRPD of crystalline erlotinib hydrochloride Form B and the associated values, respectively, prepared as per Example 7.

FIG. 8a and FIG. 8b depicts XRPD of crystalline erlotinib hydrochloride Form B and the associated values, respectively, prepared as per Example 8.

FIG. 9a and FIG. 9b depicts XRPD of crystalline erlotinib hydrochloride Form B and the associated values, respectively, prepared as per Example 9.

FIG. 10a and FIG. 10b depicts XRPD of crystalline erlotinib hydrochloride Form B and the associated values, respectively, prepared as per Example 10.

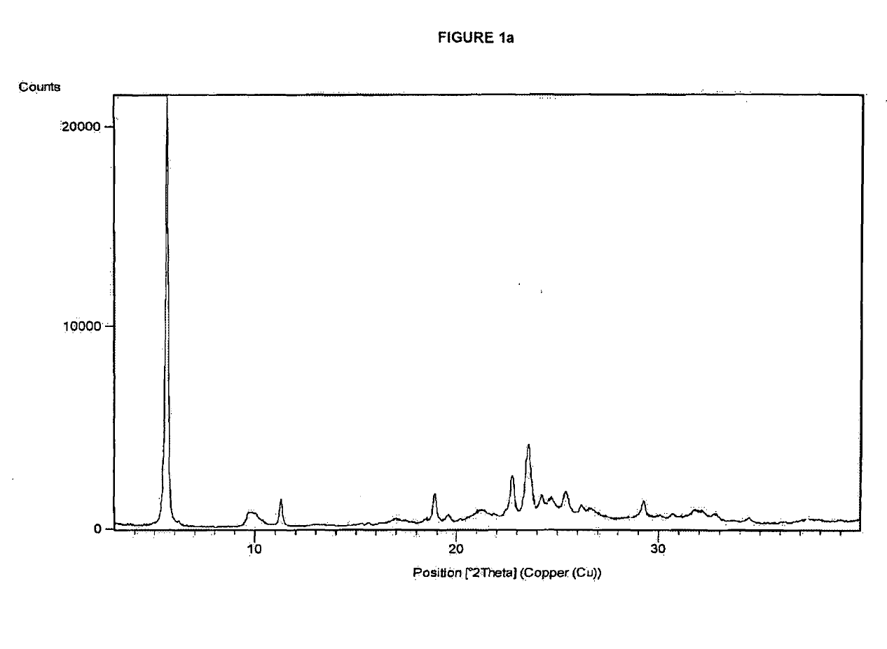

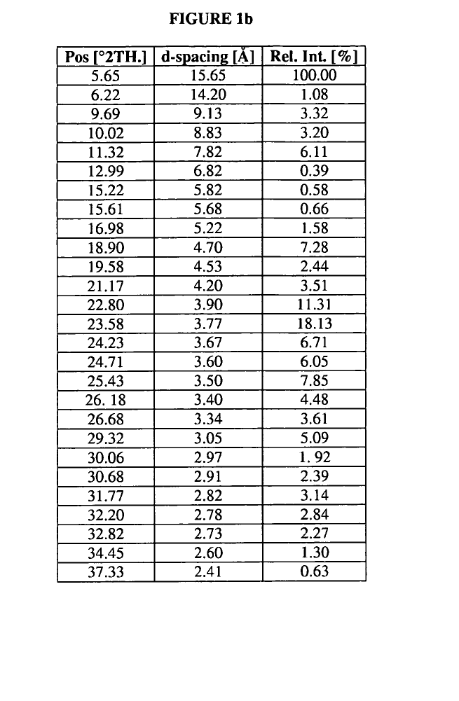

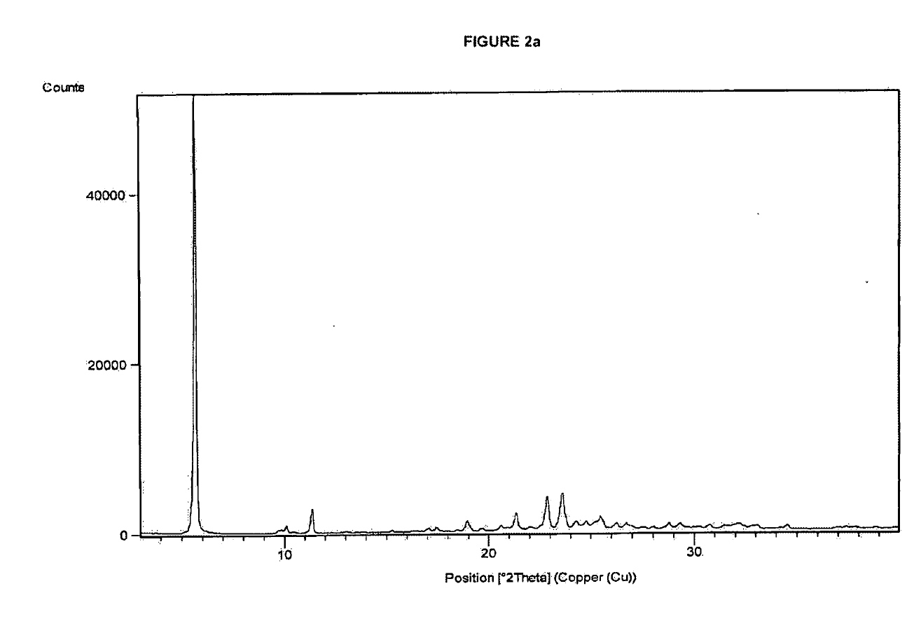

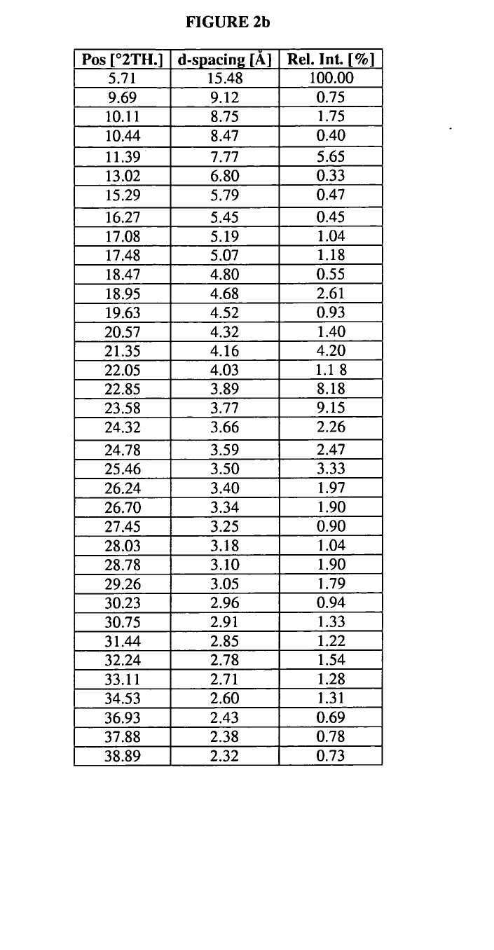

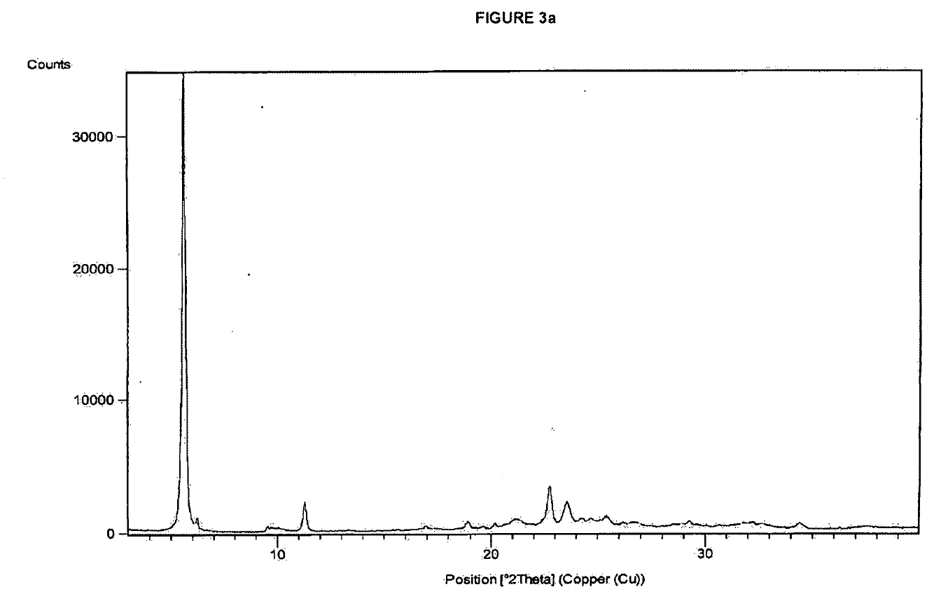

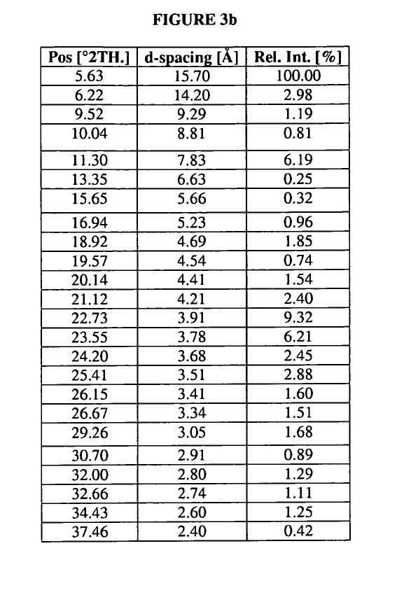

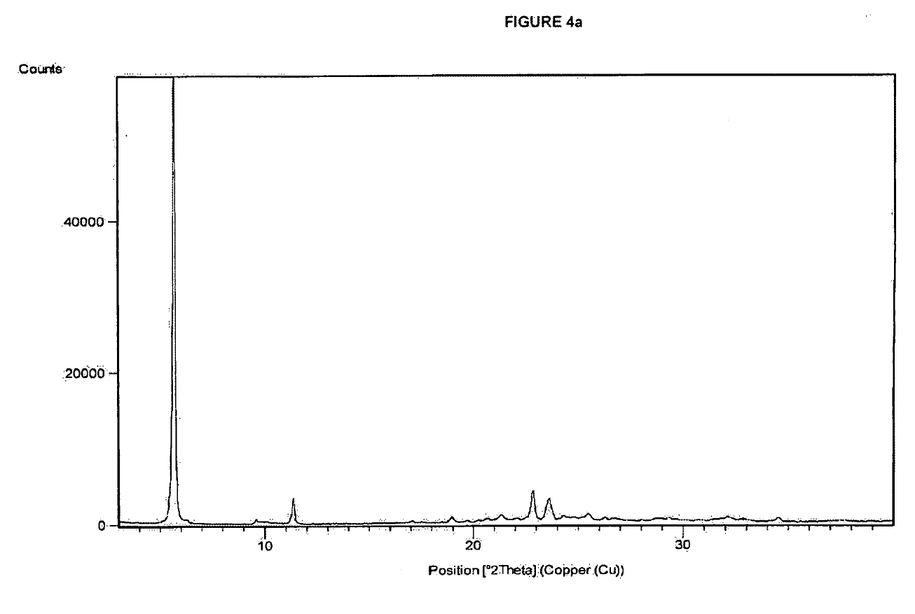

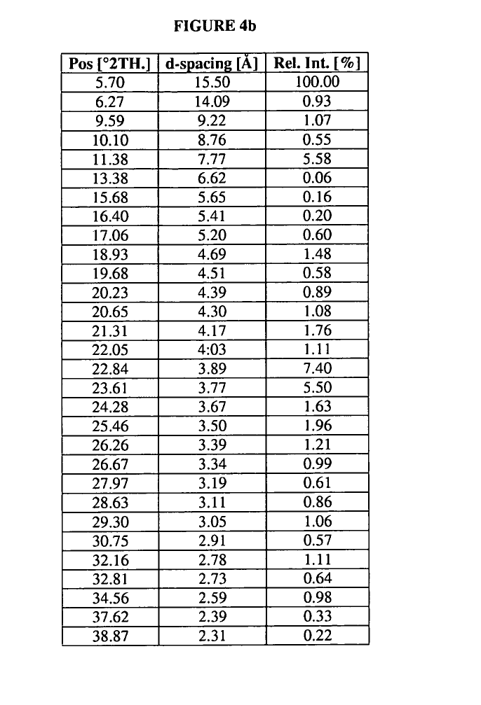

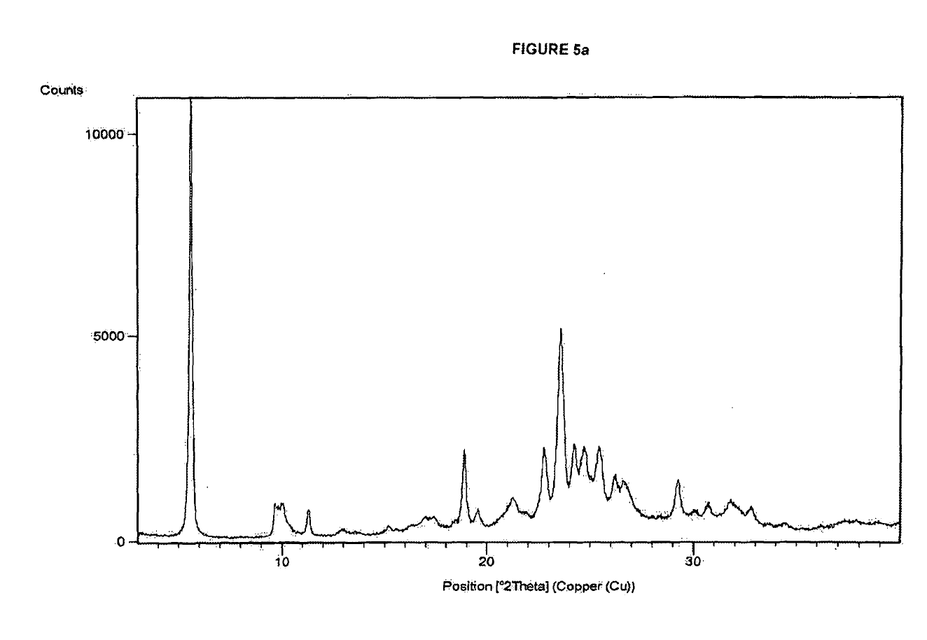

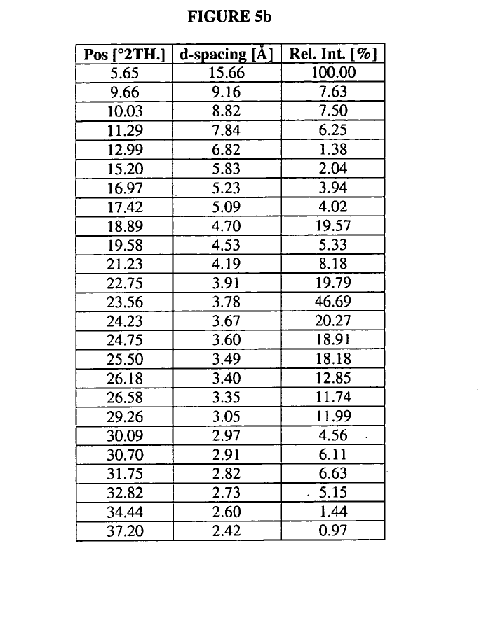

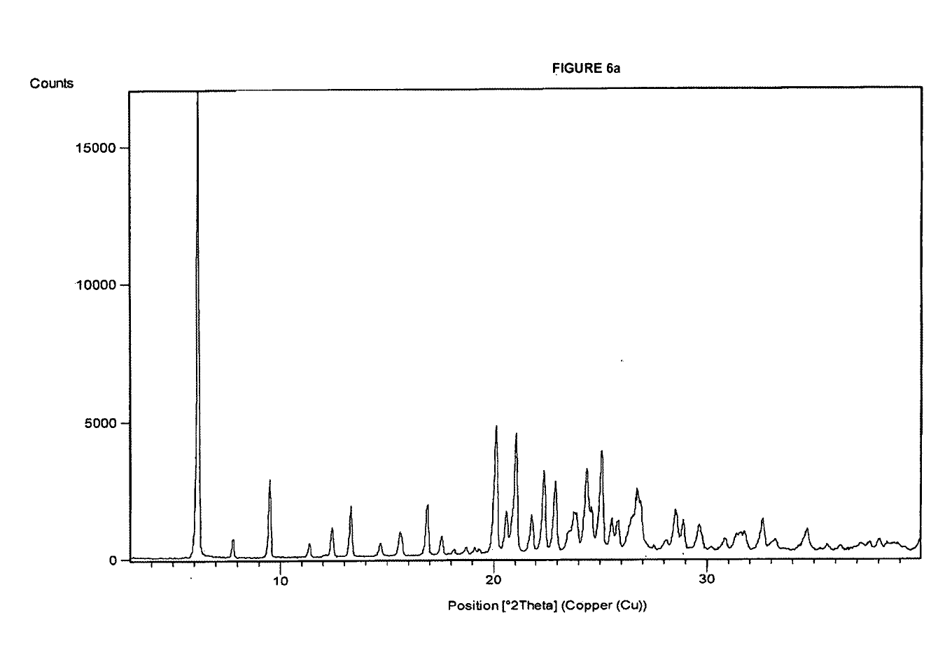

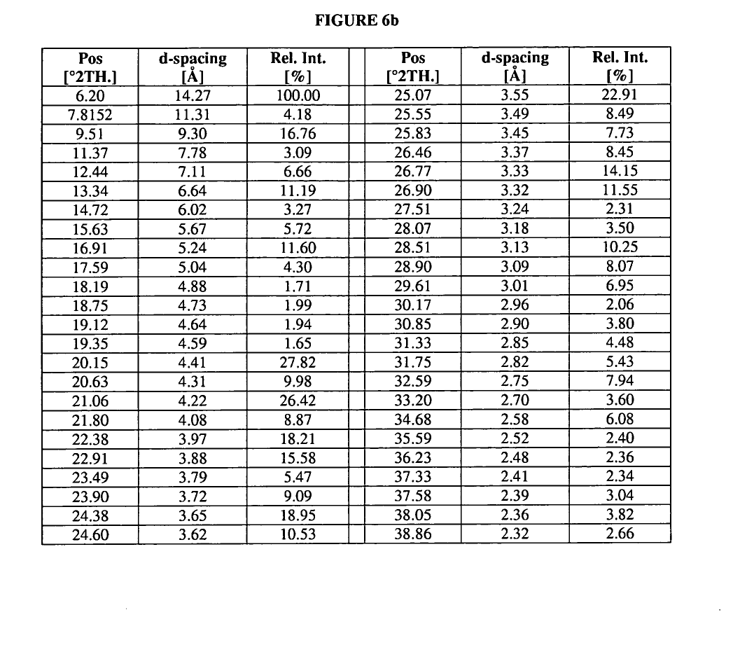

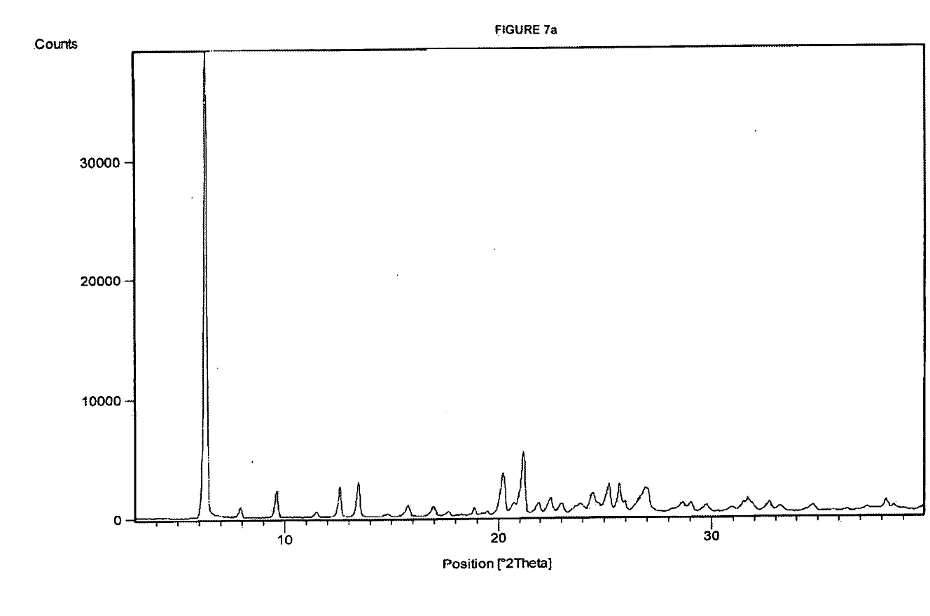

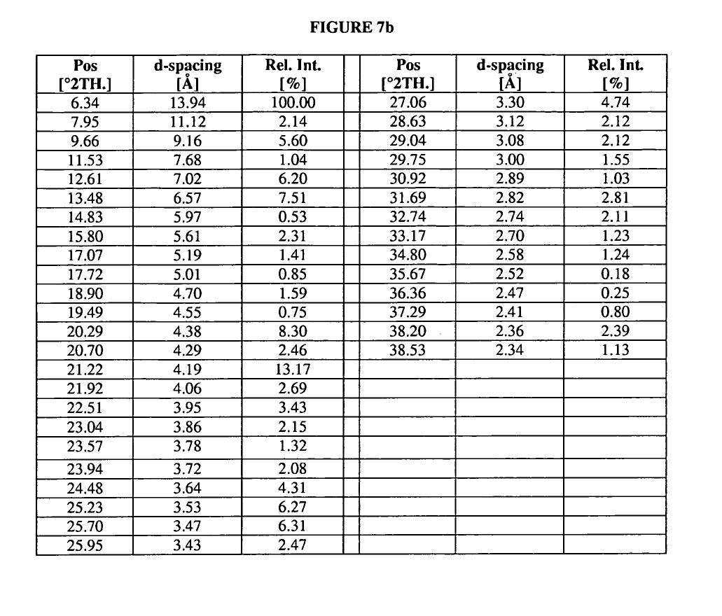

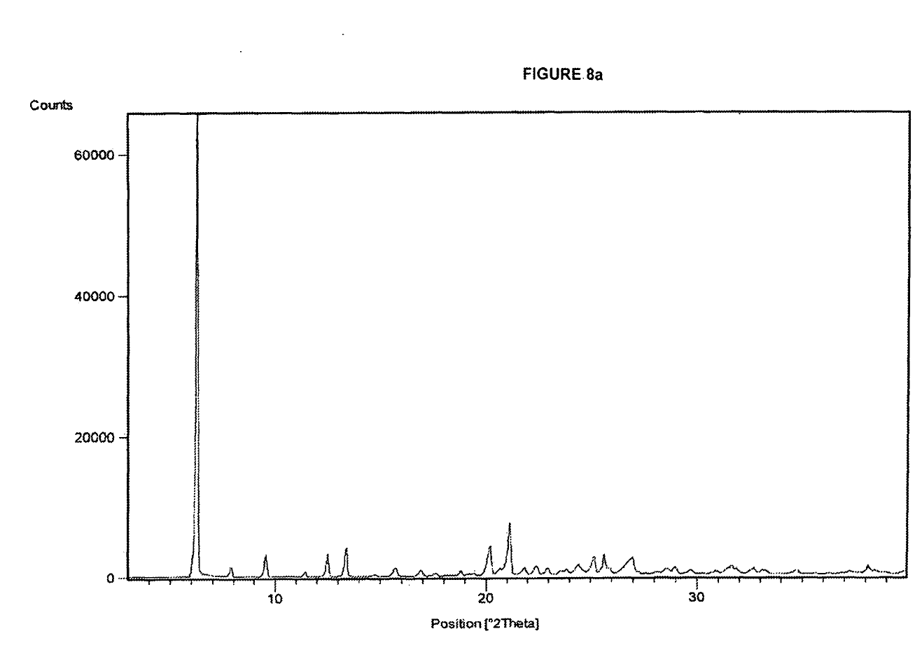

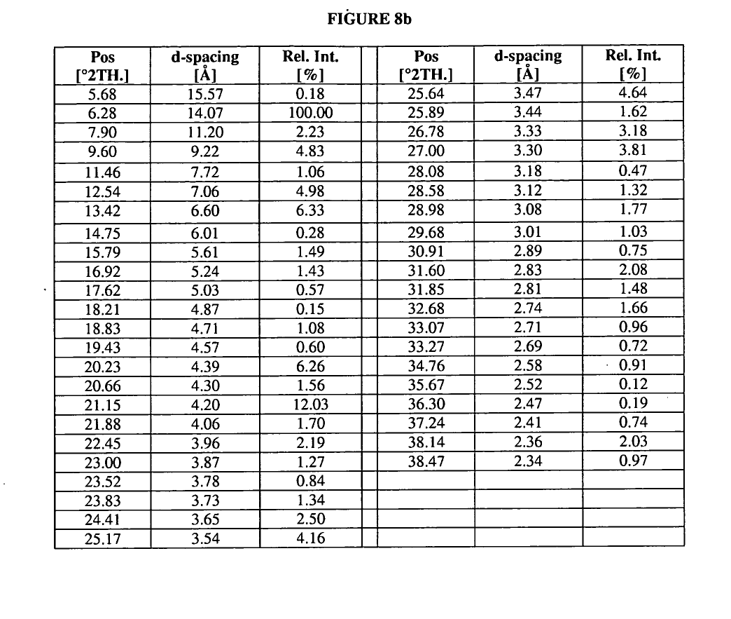

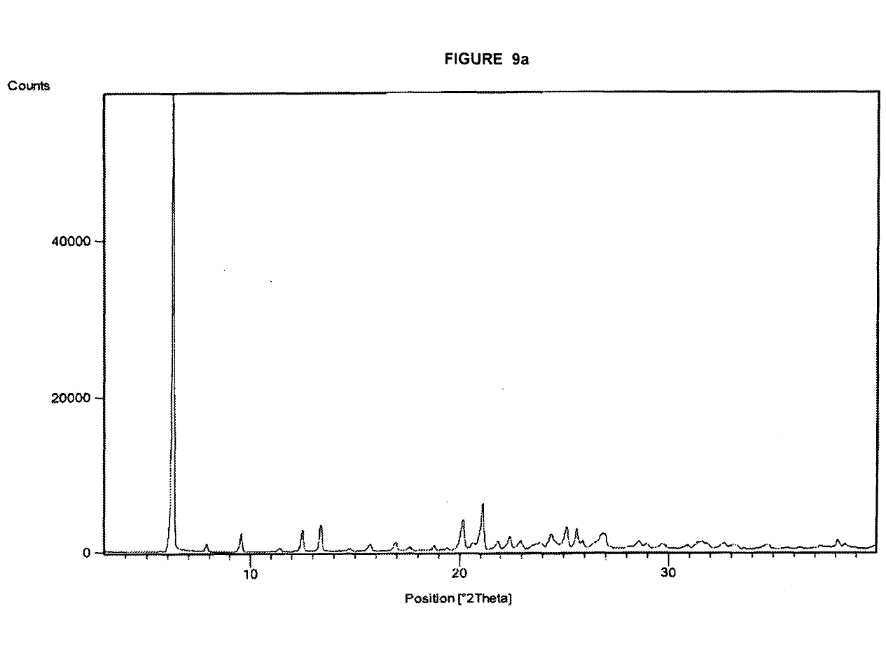

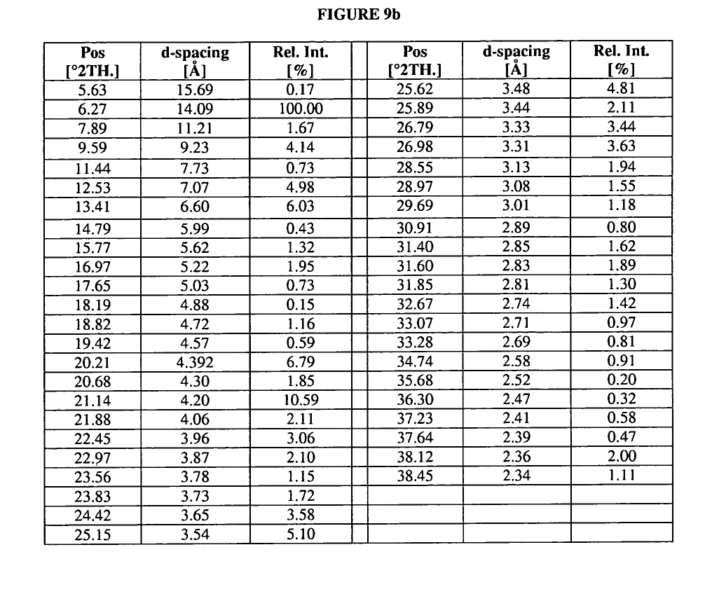

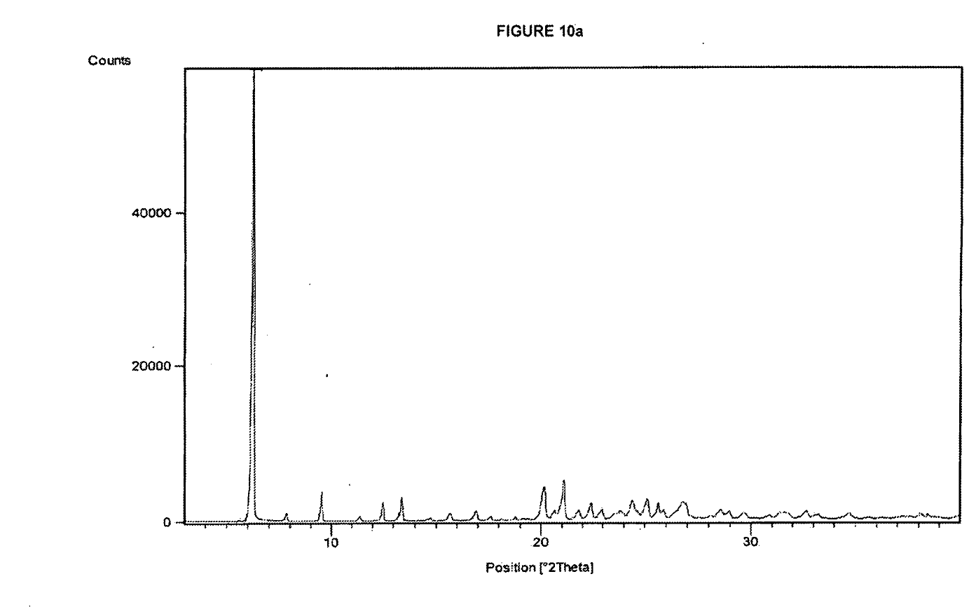

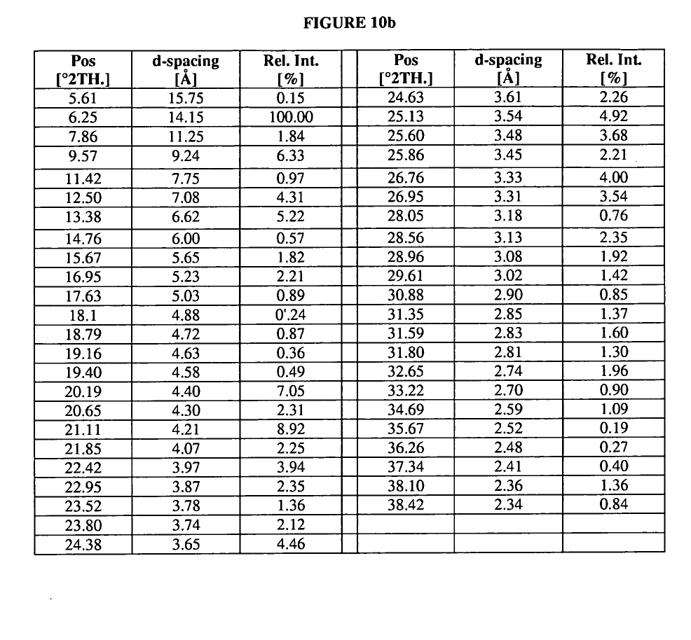

The X-ray powder diffractograms (XRPD) of the samples were determined by using Instrument: PANalytical; Mode: Expert PRO; Detector: Xcelerator; ScanRange: 3-40; Step size: 0.02; Range: 3-40 degree 2 theta; CuKα radiation at 45 kV.

While the present invention has been described in terms of its specific embodiments, certain modifications and equivalents will be apparent to those skilled in the art and are intended to be included within the scope of the present invention.

### EXAMPLES

### Example 1

### Preparation of Erlotinib Hydrochloride Form A

Erlotinib base (2.0 g) was charged into acetone (50 ml) at 25° C. to 30° C. and stirred for 10 minutes at 25° C. to 30° C. to get a clear solution. The solution was cooled to 15° C. to 20° C. and anhydrous hydrogen chloride gas was passed until crystallization appeared at 15° C. to 20° C. The temperature of the reaction mass was raised to 25° C. to 30° C. and stirred for 3 hours at 25° C. to 30° C. The reaction mass was filtered, washed with acetone (10 ml) and dried under vacuum for 6 hours at 40° C. to 45° C. to obtain the title compound.

Yield: 2.05 g (94%)

### Example 2

### Preparation of Erlotinib Hydrochloride Form A

Erlotinib base (2.0 g) was charged into dichloromethane (80 ml) at 25° C. to 30° C. and stirred for 10 minutes at 25° C. to 30° C. to give a clear solution. The solution was cooled to 15° C. to 20° C. and anhydrous hydrogen chloride gas was passed until crystallization appeared at 15° C. to 20° C. The temperature of the reaction mass was raised to 25° C. to 30° C. and stirred for 3 hours at 25° C. to 30° C. The reaction mass was filtered, washed with dichloromethane (10 ml) and dried under vacuum for 6 hours at 40° C. to 45° C.

Yield: 1.95 g (89%)

### Example 3

### Preparation of Erlotinib Hydrochloride Form A

Erlotinib base (2.0 g) was charged into ethyl acetate (30 ml) at 25° C. to 30° C. and stirred for 10 minutes at 65° C. to 70° C. to give a clear solution. Anhydrous hydrogen chloride gas was passed until crystallization appeared at 55° C. to 60° C. The temperature of the reaction mass was cooled to 25° C. to 30° C. and stirred for 3 hours at 25° C. to 30° C. The reaction mass was filtered, washed with ethyl acetate and dried under vacuum for 6 hours at 40° C. to 45° C.

Yield: 0.8 g (36%)

### Example 4

### Preparation of Erlotinib Hydrochloride Form A

Erlotinib base (2.0 g) was charged into dichloromethane (40 ml) at 25° C. to 30° C. and stirred for 10 minutes at 25° C. to 30° C. to give a clear solution. The solution was cooled to 15° C. to 20° C. and 4% hydrogen chloride in ether (10 ml) was added slowly in 5 minutes at 15° C. to 20° C. The temperature of the reaction mass was raised to 25° C. to 30° C. and stirred for 3 hours at 25° C. to 30° C. The reaction mass was filtered, washed with dichloromethane (10 ml) and dried under vacuum for 6 hours at 40° C. to 45° C.

Yield: 2.18 g (100%)

### Example 5

### Preparation of Erlotinib Hydrochloride Form A

Erlotinib base (2.0 g) was charged into a mixture of dichloromethane (40 ml) and ether (15 ml) at 25° C. to 30° C. and stirred for 10 minutes at 25° C. to 30° C. to give a clear solution. The solution was cooled to 15° C. to 20° C. and 4% hydrogen chloride in ether (10 ml) was added slowly in 5 minutes at 15° C. to 20° C. The temperature of the reaction mass was raised to 25° C. to 30° C. and stirred for 3 hours at 25° C. to 30° C. The reaction mass was filtered, washed with dichloromethane (6.5 ml) and ether (3.5 ml) and dried under vacuum for 6 hours at 40° C. to 45° C.

Yield: 2.1 g (97%)

### Example 6

### Preparation of Erlotinib Hydrochloride Form B

Erlotinib base (2.0 g) was charged into acetonitrile (50 ml) at 25° C. to 30° C. and stirred for 10 minutes at 25° C. to 30° C. to get uniform slurry. Anhydrous hydrogen chloride gas was passed until crystallization appeared at 25° C. to 30° C. The reaction mass was stirred for 5 hours at 25° C. to 30° C. The reaction mass was filtered, washed with acetonitrile (10 ml) and dried under vacuum for 16 hours at 40° C. to 45° C. to obtain the title compound.

Yield: 1.92 g (88%)

### Example 7

### Preparation of Erlotinib Hydrochloride Form B

Erlotinib hydrochloride (5.0 g) was charged in to a mixture of acetonitrile, water mixture (2:1, 125 ml) at 25° C. to 30° C. The reaction mass was stirred at 70° C. to 75° C. for about 10 minutes to get a clear solution. The solution was cooled to 25° C. to 30° C. and stirred for 17 hours at 25° C. to 30° C. The product obtained was filtered, washed with acetonitrile, water mixture (2:1, 1 x 10m1), suck dried and dried under vacuum for about 17 hours at 40° C. to 45° C. to give erlotinib hydrochloride Form B.

Yield: 2.2 g (44%)

### Example 8

### Preparation of Erlotinib Hydrochloride Form B

Erlotinib hydrochloride (5.0 g) was charged in to a mixture of acetonitrile, water mixture (2:1, 125 ml) at 25° C. to 30° C. The reaction mass was stirred for 10 minutes at 70° C. to 75° C. to get a clear solution. The obtained solution was cooled to 25° C. to 30° C. and stirred for 17 hours at 25° C. to 30° C. The reaction mass was further cooled to 0° C. to 5° C. and stirred for 2 hours to 3 hours at 0° C. to 5° C. The obtained product was filtered, washed with acetonitrile, water mixture (2:1, 1×10 ml), suck dried and dried under vacuum for about 17 hours at 40° C. to 45° C. to give erlotinib hydrochloride Form B.

Yield: 3.95 g (79%)

### Example 9

### Preparation of Erlotinib Hydrochloride Form B

Erlotinib hydrochloride (5.0 g) was charged in to a mixture of acetone, water mixture (2:1, 125 ml) at 25° C. to 30° C. The reaction mass was stirred for 10 minutes at 70° C. to 75° C. to get a clear solution. The solution was cooled to 25° C. to 30° C. and stirred for 17 hours at 25° C. to 30° C. The obtained product was filtered, washed with acetone, water mixture (2:1, 1×10ml), suck dried and dried under vacuum for about 17 hours at 40° C. to 45° C. to give erlotinib hydrochloride Form B.

Yield: 2.83 g (56%)

### Example 10

### Preparation of Erlotinib Hydrochloride Form B

Erlotinib hydrochloride (5.0 g) was charged in to a mixture of acetone, water mixture (2:1, 125 ml) at 25° C. to 30° C. The reaction mass was stirred for 10 minutes at 70° C. to 75° C. to get a clear solution. The obtained solution was cooled to 25° C. to 30° C. and stirred for 17 hours at 25° C. to 30° C. The reaction mass was further cooled to 0° C. to 5° C. and stirred for 2 hours to 3 hours at 0° C. to 5° C. The obtained product was filtered, washed with acetone, water mixture (2:1, 1×10 ml), suck dried and dried under vacuum for about 17 hours at 40° C. to 45° C. to give erlotinib hydrochloride Form B.

Yield: 4.43 g (88%)

We claim:

1. A process for preparing erlotinib hydrochloride Form A which comprises of:
a) providing a mixture comprising erlotinib and a solvent selected from acetone, dichloromethane, or a mixture thereof;
b) treating the mixture obtained in step a) with hydrogen chloride gas; and
c) isolating crystalline erlotinib hydrochloride Form A.

2. A process according to claim 1, wherein the mixture of step a) is stirred at a temperature of about 25° C. to about 30° C.

3. A process according to claim 1, wherein the mixture of step a) is treated with hydrogen chloride gas at a temperature of about 15° C. to about 20° C.

4. A process according to claim 3, wherein the step b) further comprises raising the temperature of the reaction mass to about 25° C. to about 30° C.

5. A process according to claim 4, wherein the step b) further comprises stirring the reaction mass at about 25° C. to about 30° C. for about 3 hours to about 4 hours.

6. A process according to claim 1, wherein the isolation of erlotinib hydrochloride Form A is carried out by filtration, solvent removal, layer separation, centrifugation, concentration, distillation, or a combination thereof.

7. A process for preparing erlotinib hydrochloride Form A which comprises of:
a) providing a mixture comprising erlotinib and a solvent selected from dichloromethane, ether or mixture thereof;
b) treating the mixture obtained in step a) with 4% hydrogen chloride in ether; and
c) isolating crystalline erlotinib hydrochloride Form A.

8. A process according to claim 7, wherein the mixture of step a) is stirred at a temperature of about 25° C. to about 30° C.

9. A process according to claim 7, wherein the mixture of step a) is treated with 4% hydrogen chloride in ether at a temperature of about 15° C. to about 20° C.

10. A process according to claim 9, wherein the step b) further comprises raising the temperature of the reaction mass to about 25° C. to about 30° C.

11. A process according to claim 9, wherein the step b) further comprises stirring the reaction mass at about 25° C. to about 30° C. for about 3 hours to about 4 hours.

12. A process according to claim 7, wherein the isolation of erlotinib hydrochloride Form A is carried out by filtration, solvent removal, layer separation, centrifugation, concentration, distillation, or a combination thereof.

13. A process for preparing erlotinib hydrochloride Form A which comprises of:
a) providing a mixture comprising erlotinib and ethyl acetate;
b) treating the mixture obtained in step a) with hydrogen chloride gas at about 40° C. to about 70° C.; and
c) isolating crystalline erlotinib hydrochloride Form A.

14. A process according to claim 13, wherein the mixture obtained in step a) is treated with hydrogen chloride gas at about 55° C. to about 60° C.

15. A process according to claim 13, wherein the step b) further comprises cooling the reaction mass to a temperature of about 25° C. to about 30° C.

16. A process according to claim 15, wherein the step b) further comprises stirring the reaction mass at about 25° C. to about 30° C. for about 3 hours to about 4 hours.

17. A process according to claim 13, wherein the isolation of erlotinib hydrochloride Form A is carried out by filtration, solvent removal, layer separation, centrifugation, concentration, distillation, or a combination thereof.

18. A process for preparing erlotinib hydrochloride Form B which comprises of:
a) providing a mixture comprising erlotinib and acetonitrile;
b) treating the mixture obtained in step a) with hydrogen chloride gas; and
c) isolating crystalline erlotinib hydrochloride Form B.

19. A process according to claim 18, wherein the mixture of step a) is stirred at a temperature of about 25° C. to about 30° C.

20. A process according to claim 18, wherein the mixture obtained in step a) is treated with hydrogen chloride gas at about 0° C. to about 80° C.

21. A process according to claim 20, wherein the mixture obtained in step a) is treated with hydrogen chloride gas at about 25° C. to about 30° C.

22. A process according to claim 18, wherein the step b) further comprises stirring the reaction mass at about 25° C. to about 30° C. for about 5 hours.

23. A process according to claim 18, wherein the isolation of erlotinib hydrochloride Form B is carried out by filtration, solvent removal, layer separation, centrifugation, concentration, distillation, or a combination thereof.

24. A process for preparing erlotinib hydrochloride Form B which comprises of:
a) dissolving erlotinib hydrochloride in a solvent mixture selected from acetone/water and acetonitrile/water;
b) cooling the solution obtained in step a); and
c) isolating crystalline erlotinib hydrochloride Form B.

25. A process according to claim 24, wherein dissolving erlotinib hydrochloride in a solvent mixture selected from acetone/water and acetonitrile/water is carried out at a temperature of about 45° C. to about 80° C.

26. A process according to claim 24, wherein cooling the solution obtained in step a) is carried out at a temperature about 25° C. to about 30° C.

27. A process according to claim 26, wherein the step b) further comprises stirring the reaction mass at about 25° C. to about 30° C. for about 1 hour to about 24 hours.

28. A process according to claim 26, wherein the step b) further comprises stirring the reaction mass at about 0° C. to about 5° C. for about 2 hours to about 3 hours.

29. A process according to claim 24, wherein the isolation of erlotinib hydrochloride Form B is carried out by filtration, solvent removal, layer separation, centrifugation, concentration, distillation, or a combination thereof.
